(19) United States
(12) Patent Application Publication
(10) Pub. No.: US 2009/0306046 A1
(43) Pub. Date: Dec. 10, 2009

(54) METHOD OF TREATMENT OF HORMONE DEPLETION INDUCED VASOMOTOR SYMPTOMS

(75) Inventor: Edward Baker, Branchburg, NJ (US)

Correspondence Address: ORGANON USA, INC.;c/o Schering-Plough Corporation, 2000 Galloping Hill Road, Mail Stop: K-6-1, 1990, Kenilworth, NJ, 07033, US

(73) Assignee: N.V. ORGANON, OSS, NL (unknown)

(21) Appl. No.: 11/917,456

(22) PCT Filed: Jun. 21, 2006
(86) PCT No.: PCT/US06/24111

§ 371 Date: Dec. 13, 2007

Related U.S. Application Data
(60) Provisional application No. 60/694,335, filed on Jun. 27, 2005.

(51) Int. Cl. A61K 31/55; A61P 5/00

(52) U.S. Cl. 514/214.02

(57) ABSTRACT

The invention provides a method to effectively wean a woman from hormone therapy for treatment of vasomotor symptoms by reducing the dosage of the hormonal agent to zero, while a non-hormonal drug in an amount, which is therapeutically effective for treating vasomotor symptoms, is administered or initiated and continued for a limited period of a duration between 3 and 8 months.

The present application claims priority of U.S. Provisional Patent Application No. 60/694,355, filed on Jun. 27, 2006.

The invention pertains to a method of treatment of hormone depletion induced vasomotor symptoms by using administration of non-hormonal drug therapy. At a recent NIH sponsored meeting which reviewed the “state of the science for the management of menopause related symptoms” in March 2005 it was concluded that: “Based on review of currently available cohort and cross-sectional population studies, vasomotor symptoms and vaginal dryness are symptoms most consistently associated with the menopausal transition. Sleep disturbance, somatic complaints, urinary complaints, sexual dysfunction, mood, and quality of life are inconsistently associated.” and: “Estrogen, in either opposed or unopposed regimens, is the most consistently effective therapy for vasomotor symptoms, and demonstrates benefit in most trials evaluating urogenital symptoms. Some, but not all, trials evaluating sleep, mood and depression, sexual function, and quality of life outcomes also report benefit with estrogen compared to placebo.” and: “For women with breast cancer, results of 15 randomized controlled trials indicate that clonidine, venlafaxine, and megestrol acetate are associated with significantly improved measures of hot flashes, and vitamin E, black cohosh, isoflavones, magnets, and fluoxetine are not. Results for nonvasomotor outcomes are mixed.” and: “in order to fill evidence gaps, future research could focus on: [- -] Trials demonstrating how to discontinue estrogen when symptoms subside, including the effectiveness of tapering doses and/or replacing with other therapies including no n-drug interventions.”

Clearly there is much to be discovered about how vasomotor symptoms work and what to optimally do about them. Currently, the gold standard for reduction of vasomotor symptoms associated with menopause is estrogen therapy. European and American regulatory authorities, and groups such as the American College of Obstetrics and Gynecology and the North American Menopause Society all currently recommend using the lowest appropriate estrogen dose and limiting the length of therapy to that which is necessary to meet treatment goals. What is lacking is advice on how to wean a patient from estrogen therapy.

It could be that estrogen therapy simply delays the hot flashes a patient experiences at the time of menopause to a time when hormone therapy is discontinued. This is supported by the experience of many following the sudden self-imposed withdrawal of women from hormone therapy in light of the recent reports regarding outcomes from the Women's Health Initiative in 2002. Many of these women experienced significant vasomotor symptoms to the extent that they eventually restarted estrogen therapy. Many women will be able to discontinue hormones on their own, while others will have substantial symptoms from estrogen withdrawal and will either be successful quitters, but suffer or will return to therapy. These are the two populations that will benefit from our invention. (See D. Grady, Obstet Gynecol 2003:102 (6); 1233-1239).

It is a long-standing desire to be able to help patients quit hormones. The use of paroxetine and various other selective serotonin re-uptake inhibitors as an alternative to hormonal therapy are well known to the average gynecologist in the US. Yet despite this, it had not been thought of to use these drugs to wean a patient from hormonal therapy. In the NIH meeting mentioned above, options of either weaning a patient from estrogen, over some course of time, or weaning a patient to another medication, which she would be able to take with less risk, was considered, but there was no thought of using an agent to assist in moving a patient from estrogen to no therapy whatsoever.

The present invention provides a method to effectively wean a woman from hormone therapy for treatment of vasomotor symptoms by reducing the dosage of the hormonal agent to zero, while a non-hormonal drug in an amount, which is therapeutically effective for treating vasomotor symptoms, is administered or initiated and continued for a limited period of a duration between 3 and 8 months. In a more specific embodiment of the invention the duration of the limited period is between 4 and 8 months and in another more specific embodiment the duration is between 5 and 7 months.

Without being bound by any theory it seems that an explanation for the method is that a protracted time is needed to adjust to lower levels of hormonal action, whereby the frequency and severity of vasomotor symptoms decreases.

The success rate of the weaning method can be further improved by selecting postmenopausal women. This characteristic can be established according to the usual manners available to the clinician, such as the time period lapsed since the last menstruation, the age of the woman, in particular an age of 65 and older commonly indicates postmenopausal state, etc. The determination can be verified after weaning by measuring endogenous FSH (follicle stimulating hormone), which will no longer be fluctuating, but rather be a constant high plasma level (>40 mIU/mL).

The terms in this description are used with the following meaning:
“non-hormonal” is a mechanism of action of a drug therapy of vasomotor symptoms not based on activation of the estrogen receptor.
“weaning” is the termination of hormone therapy.
“weaning agent” is a non-hormonal drug given to assist weaning
“hormone therapy” is a treatment against undesirable effects caused by decline in endogenous estrogens in a woman based on reinstatement of activation of estrogen receptors, for example by estrogen therapy or hormone replacement therapy or prescription of regimes to maintain the monthly cycle in a woman.
“non-hormonal drug” is a drug not having a hormonal mechanism of therapeutic action.
“non-hormonal drug therapy” is a therapy for countering one or more undesirable effects caused by decline in endogenous estrogens with a non-hormonal agent.
“A therapeutically effective amount of a non-hormonal drug” is an amount of the non-hormonal drug which prevents to a large extent one or more of the undesirable effects caused by decline in endogenous estrogens in a woman.
“Hot flash” is a sensation of heat or burning which usually starts in the upper torso and head. It is probably the most distressing symptom of menopause and is experienced by approximately 80% of menopausal women.
“Menopause”—the final menstrual period, usually diagnosed retrospectively after at least one year without menstruation. It is, though, commonly understood, and used here in that sense, that the expression ‘menopausal women’ refers to women who are in a period of their life that is transitional between mature female physiological functioning and postmenopausal functioning. In that sense the term ‘vasomotor symptoms (hot flashes) associated with menopause can be understood.

It is an essential means of the invention that the dosage of the hormonal agent is reduced while giving or initiating non-hormonal therapy for vasomotor symptoms. The reduction in dosing of the hormonal treatment can be immediate by termination of any administration of the hormonal agent or gradual over a period of at most two weeks, during which the dosage is reduced stepwise. If administration of non-hormonal therapy has not already been started shortly, that is a few days, before reduction of the hormonal therapy, the non-hormonal therapy should start within a few days, at most four days after having stopped the administration of a hormonal agent.

As compound for non-hormonal treatment any compound effective against hot flash can be selected, such as mirtazapine. For the treatment of menopausal symptoms mirtazapine is to be administered to a women in a suitable daily dose, which will be in the range of from 0.5 to 140 mg, calculated on the weight content of base, per recipient per day, preferably in the range of 1 to 20 mg and most preferably in the lower range of 1-10 mg or even below 5 mg of the base per recipient per day. In general, parenteral administration requires lower dosages than other methods of administration which are more dependent upon absorption. However, the daily dosages are between 0.01 and 1.5 mg/kg body weight of the recipient.

In the case of tolerance development, treatments can be further optimalized by increasing the dose up to 5 times in the course of a chronic treatment in humans. The desired dose may be presented as one, two, three or more sub-doses administered at appropriate intervals throughout the day.

Mirtazapine is known to existing in two enantiomers in S- or R- configuration. The drug can be used for the purpose of the invention as racemic mixture or as one enantiomer substantially free of the other enantiomer. The S-mirtazapine is preferred as active ingredient for the method according to the invention. The compound can be used for the purpose according to the invention as a free base or as one or more of the commonly accepted acid addition salts. Such compounds can be used in pure form or in admixture with pharmaceutical excipients.

The amount of mirtazapine, S-mirtazapine or R-mirtazapine, also referred to herein as the active ingredient, which is required to achieve a therapeutic effect will, of course, vary with the particular compound, the route of administration and the age and other conditions of the recipient. The amounts of mirtazapine defined in this description refer to the amount of free base of mirtazapine, unless indicated otherwise.

While it is possible for the active ingredient to be administered alone, it is preferable to present it as a pharmaceutical formulation. Accordingly, the present invention further provides a pharmaceutical formulation for use in the treatment according to the invention, together with a pharmaceutically acceptable carrier thereof and optionally other therapeutic agents. The carrier must be “acceptable” in the sense of being compatible with the other ingredients of the formulation and not deleterious to the recipients thereof. Suitable excipients are made available e.g., in the Handbook of Pharmaceutical Excipients, 2^{nd} Edition; Editors A. Wade and P. J. Weller, American Pharmaceutical Association, Washington, The Pharmaceutical Press, London, 1994. The invention further includes a pharmaceutical formulation, as hereinbefore described, in combination with packaging material suitable for the pharmaceutical formulation, said packaging material including instructions for the use of the pharmaceutical formulation in the treatment of hot flush.

Formulations include those suitable for oral or vaginal administration. The formulations may be prepared by any methods well known in the art of pharmacy. Such methods include the step of bringing into association the active ingredient with the carrier which constitutes one or more accessory ingredients. Such accessory ingredients include those conventional in the art, such as, fillers, binders, diluents, disintegrants, lubricants, colorants, flavoring agents and wetting agents.

Formulations suitable for oral administration may be presented as discrete units such as tablets or capsules each containing a predetermined amount of active ingredient; as a powder or granulates; as a solution or suspension. The active ingredient may also be presented as a bolus or paste, or may be contained within liposomes or microparticles.

Formulations, which are parenteral (for example subcutaneous) may also be presented in a suitable sustained release form.

Mirtazapine, S-mirtazapine and R-mirtazapine can be prepared in several manners, Mirtazapine may be prepared using the method described in U.S. Pat. No. 4,062,848, possibly followed by purification to an enantiomerically pure form. Enantiomerically pure mirtazapine can also be obtained by stereoselective synthesis (WO 2005/005410).

The following examples are for illustration and should not be considered to be limiting in anyway:

### EXAMPLE

Mirtazapine is used as non-hormonal therapy for vasomotor symptoms. Treatment dose is 15 mg. The length of weaning is 6 months.

In this example subjects for treatment have the following characteristics: Postmenopausal women of 60-65 year old who have been on hormone therapy for the treatment of vasomotor symptoms associated with menopause for at least 2 years. They have either a need or a desire to discontinue hormone therapy. They are informed of the risks of mirtazapine. They are informed that successful use of mirtazapine for the relief of vasomotor symptoms associated with menopause has been reported in the medical literature. This method should not be used for women who have a condition or take medications that preclude the use of mirtazapine.

A woman treated according to this example is instructed to take 15 mg mirtazapine by mouth on the day that she is to discontinue hormone therapy. Mirtazapine is prescribed to be taken at bedtime and after all her evening rituals are done so that she can lay down right after taking mirtazapine. She is instructed to continue taking 15 mg of mirtazapine at bedtime for 6 months after which mirtazapine administration is discontinued. After 6 months treatment is discontinued with the benefit that withdrawal vasomotor symptoms are absent or strongly reduced in comparison to women on hormone therapy.

Method to measure hot flash frequency: by self rating or according to the method described by Freedman et al J. Clin Endocrin & Metabolism, Vol 80, pp 2354-2358.

1. A method to effectively wean a woman from hormone therapy for treatment of vasomotor symptoms by reducing the dosage of the hormonal agent to zero, while a non-hormonal drug in an amount, which is therapeutically effective for treating vasomotor symptoms, is administered or initiated and continued for a limited period of a duration between 3 and 8 months.

2. The method according to claim 1, wherein the non-hormonal drug is S-mirtazapine or a salt thereof.

3. The method according to claim 1, wherein the woman is after her menopause.
